(19) United States
(12) Patent Application Publication
(10) Pub. No.: US 2005/0271640 A1
(43) Pub. Date: Dec. 8, 2005

(54) Method for inhibiting yeast growth

(76) Inventors: Tarja Suomalainen, Helsinki (FI); Annika Mayra-Makinen, Helsinki (FI)

Correspondence Address: NIXON & VANDERHYE, PC, 901 NORTH GLEBE ROAD, 11TH FLOOR, ARLINGTON, VA, 22203, US

(21) Appl. No.: 10/533,816

(22) PCT Filed: Nov. 3, 2003
(86) PCT No.: PCT/FI03/00813

§ 371 Date: May 3, 2005

(30) Foreign Application Priority Data
Nov. 4, 2002 (FI) 20021938

(51) Int. Cl. A61K 45/00; A61K 47/00

(52) U.S. Cl. 424/93.45; 424/439

(57) ABSTRACT

The invention relates to the use of microbes Lactobacillus rhamnosus LGG, ATCC 53103, Latobacillus rhamnosus LC705, DSM 7061, and Propionibacterium freudenreichii ssp. shermanii PJS, DSM 7067 in inhibiting yeast growth, for preventing and treating diseases caused by yeast and for relieving yeast-related symptoms in animals or humans.

### FIELD OF THE INVENTION

The invention relates to inhibiting the growth of yeasts. In particular, products and methods for inhibiting the growth of yeasts and for preventing and treating diseases caused by yeasts are disclosed.

### BACKGROUND OF THE INVENTION

Yeasts are constantly present in the living environment and the organ system of humans. Even a healthy individual has Candida albicans yeast growing on the mucosal membrane and in the entire area of the gastroin-testinal tract (Shay K, Truhlar M R, Renner R P. Oropharyngeal candidosis in the older people. J Am Geriatr Soc 1997;45:863-870). The oral cavity and the tissue between teeth also provide an excellent growth medium for a number of species of microbes, among which can be found for example the Candida albicans species and, to a lesser amount, C. glabrata and C. tropicalis yeasts. Normally yeast cells are in a passive state and their growth does not harm healthy individuals. In unfavourable conditions yeasts, such as C. albicans, begin to form hyphae and thereby penetrate deeper into the mucosal membranes. This results in a local yeast infection, which in the mouth appears for example as oral candidiasis, stomatitis, or glossitis.

Yeast infections are usually preceded by reduced resistance, which may be caused by certain medication, for example use of broad-spectrum antibiotics, corticosteroids or cytostatics, by diabetes, malign tumors, or immunodeficiency. Micro-organisms of the mouth spread easily, carried by blood circulation, to other parts of the organ system, which may have severe consequences, such as septicemia, endocarditis and meningitis, particularly in persons whose general state of health has deteriorated (Shay et al. 1997).

The most common and most important cause of yeast infections in humans is Candida albicans (Mäkelä et al. 1988. L\ddot{aa}ketieteellinen mikrobiologia (Medical microbiology), 5^{th} revised edition, pp. 270-271. Published by Kustannus Oy Duodecim 1988). C. albicans can be found relatively often in the gastrointestinal tract of healthy humans: 30 to 50% carry it in the mouth and about 1% on healthy skin and in the urinary tract. Also other species of the Candida genus can be found occasionally, the most important ones being: C. tropicalis, C. pseudotropicalis, C. parapsilosis, C. knisei, and C. guilliermondi, which act as opportunistic pathogens in humans, similarly as C. albicans (Mäkelä et al. 1988).

Elderly people typically suffer from a number of diseases, which, together with the medication used for treating them, may weaken their immunity and, at the same time, their dental health (Pajukoski H, Meurman J H, Snellman-Gröhn S, Sulkava R. Oral health in hospitalized and nonhospitalized community-dwelling elderly patients. Oral Surg Oral Med Oral Pathol Oral Radiol Endod 1999:88:437443). In addition, poor dental hygiene submits elderly people to yeast infections (Budtz-Jorgensen E, Mojon P, Banon-Clement J M, Baehni P. Oral candidosis in long-term hospital care: comparison of edentulous and dentate subjects. Oral Dis 1996;2:285-290). Ageing as such increases the appearance of yeast and the amount of it, which implies that the ability of the organ system to inhibit yeast growth reduces with age (Lockhart S R, Joly S, Vargas K, Swails-Wenger J, Enger L, Soil D R. Natural defenses against Candida colonization breakdown in the oral cavities of the elderly. J Dent Res 1999;78:857-868).

In elderly people living in Helsinki, Finland, yeast growth was found in 75%, high contents being found in 33% (Närhi T O, Ainamo A, Meurman J H. Salivary yeasts, saliva, and oral mucosa in the elderly. J Dent Res 1993;72:1009-1014). Candida yeast infection, in turn, has been found in 60% of those who carry yeast (Wilkieson C, Samaranayake L P, MacFarlane T W, Larney P J, etc. Oral candidosis in the elderly in long term hospital care. J Oral Pathol Med 1991;20:13-16). The most common yeasts after Candida albicans are C. glabrata (29%), C. tropicalis (13%), Saccharomyces cerevisiae (11%), and C. parapsilosis 89%) (Lockhart et al. 1999).

One important factor increasing oral yeast growth is reduced salivation (Närhi et al. 1993). A five-year follow-up study of the effects of ageing showed that stimulated secretion of total saliva reduces with ageing, whereas buffer capacity increases (Närhi T O, Kurki N, Ainamo A. Saliva, salivary micro-organisms, and oral health in the home-dwelling old elderly—a five-year longitudinal study. J Dent Res 1999;78:1640-1646). Endocrinological diseases and abundant use of medication in particular reduce salivation (Pajukoski H, Meurman J H, Snellman-Gröhn S. Keinänen S, Sulkava R. Salivary flow and composition in elderly patients referred to an acute care geriatric ward. Oral Surg Oral Med Oral Pathol Oral Radiol Endod 1997; 84: 265-71). Yeast contents are clearly higher for those whose rate of salivation and buffer capacity has reduced (Närhi et al. 1993). The rate of salivation of elderly Finns varies between 0.6 and 1.0 ml/mm (Pajukoski et al. 1997); reduced secretion (<0.7 ml/mm) is found with as much as 55% and low buffer capacity with 34% of the elderly (Pajukoski et al. 1997).

Candidosis has conventionally been treated with antifungal agents, such as nystatin, and more severe cases with fluconazole or itraconazole (Shay et al. 1997). Also more efficient oral hygiene has enabled to reduce Candida colonization in the oral mucosal membranes of elderly confined in long-term care (Budtz-Jorgensen E, Mojon P, Rentsch A, Deslauriers N. Effects of an oral health program on the occurrence of oral candidosis on a longterm care facility. Community Dent Oral Epidemiol 2000;28:141-149). The use of chlorhexidine/xylitol chewing-gum also enabled the amount of yeast to be reduced by 22% (Simons D, Kidd E A M, Beighton D, Jones B. The effect of chlorhexidine/xylitol chewing-gum on cariogenic salivary microflora: A clinical trial in elderly patients. Caries Res 1997;31 :91-96).

Certain groups, such as patients in long-term care and elderly people, are also prone to general infections due to reduced immunity, malnutrition and chronic diseases. In addition, in elderly people oral hygiene and oral microbial flora have been found to be associated with infections of the lower respiratory ducts, such as pneumonia and bronchitis (see Scannapieco F A. Role of oral bacteria in respiratory infection. J Petiodontol 1999;70:793-802). An infection is presumably caused when pathogenic microbes are transferred through aspiration from the mouth to the respiratory ducts. The most common infections affecting patients in long-term care are: infections of the upper and lower respiratory ducts (70%), infections of the urinary tract (12%), gastroenteritis and diarrhea (12%) as well as dermatitis and infections of the soft tissue (6%) (Orr P H, Nicolle L E, Duckworth H, Brunka J, Kennedy J, Murray D et al. Febrile urinary infection in the institutionalized elderly. Am J Med 1996;100:71-77). Hygiene intervention has enabled to reduce infections of the upper respiratory ducts of elderly people in long-term hospital care by 16% (Makris A T, Morgan L, Gaber D J, Richter A, Rubino J R, Effect of comprehensive infection control program on the incidence of infections in long-term care facilities. Am J Infect Control 2000;28:3-7).

Another main area where yeast appears is the genital area; in women in particular yeast infections are common. The most common gynecological symptom is vaginitis; it is also one of the main symptoms of patients who seek medical consultation (Makela et al. 1988). Vaginitis is most commonly caused by a bacterium, the second most common cause being a yeast fungus, C. albicans and C. glabrata being the species that appear most often. Candida albicans is a yeast that belongs to the normal microflora of the vagina, and normally it does not cause infections. Usually the bacterial flora of the vagina restricts yeast growth, but in certain conditions yeast growth becomes excessive. Risk factors for a yeast infection include use of antibiotics (particularly broad-spectrum antibiotics), pregnancy, diabetes, diseases that cause immune deficiency, and use of corticosteroids. Yeast fungus can be found in 10 to 20% of patients at gynaecological outpatient departments, but only some of them become affected by a clinical vulvovaginitis caused by yeast. A basic pH favours yeast growth. The virulence of yeast depends on the yeast content, the invasiveness of its mycelium, its steroid receptors, and on the ability of yeast to form proteases, for example. Research has shown that fungal hyphae are capable of penetrating the vaginal epithelium (Mäkelä et al. 1988).

Lactic acid bacteria have been used with varying results for preventing vaginitis. Yoghurt intake for 6 months reduced Candida colonization and the occurrences of vaginitis (Hilton et al. 1992. Ingestion of yoghurt containing Lactobacillus acidophilus as prophylaxis for Candidal vaginitis. Ann Intern Med 1992;116:353-357). On the other hand, according to the research carried out by the Shalev group (1996) Lactobacillus acidophilus yoghurt, in comparison with pasteurized yoghurt, reduced vaginitis caused by bacteria, but not that caused by Candida yeast (Shalev et al. 1996. Ingestion of yoghurt containing Lactobacillus acidophilus compared with pasteurized yoghurt as prophylaxis for recurrent Candidal vaginitis and bacterial vaginosis. Arch Fam Med 1996;5.593-596; Sieber R, Dietz U-T. Lactobacillus acidophilus and yogurt in the prevention and therapy of bacterial vaginosis. Int Dairy J 1998; 8:599-607; see also Redondo-Lopez et al. 1990. Emerging role of lactobacilli in the control and maintenance of the vaginal bacterial microflora. Rev Infect Dis 1990;12:856-872).

Yeast fungus also causes urethritis.

Several studies have shown that 20-40% of people carry C. albicans in the gastrointestinal tract (Lennette et al. 1985. Manual of clinical microbiology, 4^{th} edition. American Society for Microbiology, Washington D.C., 1985). Overgrowth of yeast in the gastrointestinal tract manifests itself usually as diarrhea.

The term ‘yeast syndrome’ is usually used to refer to increased growth of Candida albicans yeast in the gastrointestinal tract, which is considered to inhibit the immune system and to cause different syndromes. Yeast overgrowth has been associated with a number of systemic symptoms, such as symptoms of the central nervous system, different aches, fatigue and symptoms of the intestines. It is assumed that the symptoms are due to toxins released by yeasts. However, there is no scientific evidence of a connection between such symptoms and yeast. Yeast syndrome is treated with a low-carbohydrate, yeast-free diet, possibly favouring soured and/or fibre-rich nutrients. Soured foods and soured dairy products in particular form an important element of the diet treatment. Consequently, lactic acid bacteria are often used to balance a disturbed intestinal flora.

Yeast thus causes many kinds of diseases, both indirectly and directly. There is therefore a constant need to find new means for inhibiting yeast growth and activity, for preventing and treating diseases caused by yeast, and for relieving yeast-related symptoms.

As an alternative to medical treatment, or in addition to it, attempts are nowadays being made to utilize other health care means as well. One of the most recent methods is to use health-promoting nutrients or natural products, which have in fact been warmly welcomed by consumers. Health-promoting products inhibiting yeast growth and activity would therefore be a highly welcome addition to the range of commercially available products. The products should preferably be pleasing and somehow familiar to the consumer, which would make them easy to adopt as a part of the normal daily diet, for example.

The effects of probiotics as anti-microbial agents have been described in the literature of the art. Lactic acid bacteria are capable of producing anti-microbial compounds, organic acids, lactic acid, fatty acids, hydrogen peroxide, diacetyl, carbon dioxide, and bacteriocins, which enables them to inhibit the growth of pathogenic microbes (McGroarty J A. Probiotic use of lactobacilli in the human female urogenital tract. FEMS Immunol Med Microbiol 1993;6:251-264). Some strains of Lactobacillus acidophilus prevented the growth of Candida in vitro by producing hydrogen peroxide (Jack M, Wood J B, Berry D R. Evidence for the involvement of thiocyanate in the inhibition of Candida albicans by Lactobacillus acidophilus. Microbios 1990;62:3746; Fitzsimmons N, Berry D R. Inhibition of Candida albicans by Lactobacillus acidophilus: evidence for the involvement of a peroxidase system. Microbios 1994;80:125-133). It has been discovered that Lactobacillus rhamnosus LGG ATCC 53103 also produces an anti-micronial compound, possibly a short-chain fatty acid, that inhibits the in vitro growth of Escheichia coli, Pseudomonas, Salmonella, Streptococcus, Bacillus, Clostridium, and Bifidobacterium, for example (Silva M, Jacobus N V, Deneke C, Gorbach S L. Antimicrobial substance from a human Lactobacillus strain. Antimicrobial Agents Chemother 1987;31:1231-1233).

Lactic acid bacteria may also prevent the adhesion of other microbes to epithelial cells. For example, some Lactobacillus acidophilus and L. casei strains have been found to prevent 22-46% of the adhesion of C. albicans to uroepithelial cells in in vitro conditions (Reid G, Tieszer C, Lam D. Influence of lactobacilli on the adhesion of Staphylococcus aureus, and Candida albicans to fibers and epithelial cells. J Indust Microbiol 1995;15:248-253). In animal tests Lactobacillus rhamnosus LGG has been discovered to reduce the amount of C. albicans in the gastrointestinal tract of mice and candidosis in the mouth area by stimulating a cell-mediated immune response to antibodies of C. albicans. (Wagner R D, Pierson C, Warner T, Dohnalek M, Farmer J. Roberts L et al. Biotherapeutic effects of probiouic bacteria on candidiasis in immunodeficient mice. Infect Immun 1997;65:41654172; Wagner R D, Pierson C, Warner T. Dohnalek M, Hilty M, Balish E. Probiotic effects of feeding heat-killed Lactobacillus acidophilus and Lactobacillus casei to Candida albicans—colonized immunodeficient mice. J Food Protect 2000;63 :638-644).

When acting together, Lactobacillus LC 705 and Propionibacterium freudenreichii ssp. shermanii PJS have been discovered to inhibit yeast growth in yoghurts and curd cheese (Suomalainen T, Mäyrä-Mäkinen A. Propionic acid bacteria as protective cultures in fermented milks and breads. Lait 1999;79:165-174).

Lactic acid bacteria and their cellular structures may activate the resistance of the organ system by increasing the activity of macrophages and natural killer cells, the amount of T and B cells and the proportion of antibodies (Perdigon G, Alvarez s, Rachid M, Agüero G, Gobbato N. Symposium: Probiotic bacteria for humans: Clinical systems for evaluation of effectiveness. Immune system stimulation by probiotics. J Dairy Sci 1995;78:1597-1606).

Lactobacillus rhamnosus LGG has also been found to enhance the normal resistance of the intestines against harmful bacteria, viruses, and yeasts (Kaila M, Isolauri E, Soppi E et al. Enchancement of the circulating antibody secreting cell response in human diarrhea by a human Lactobacillus strain. Pediatr Res 1992;32:141-144; Wagner et al. 1997). In animal tests Lactobacillus GG has also increased the content of secretory IgA in saliva (Negretti F, Casetta P, Clerici-Bagozzi D, Marini A. Researches on the intestinal and systemic immunoresponses after oral treatments with Lactobacillus GG in rabbit. Phisiopath Clin 1997;7:15-21). The secretory IgA of the mucosal membrane is known to protect the respiratory ducts, gastrointestinal tract, and urogenital organs against infections (Nagura H. Mucosal defence mechanism and secretory IgA system. Bifidobacteria Microflora 1990;9: 17-25). Lactic acid bacteria may thus also reduce infections of the respiratory tract and the gastrointestinal tract, indications of which have already been obtained in studies conducted among day-care children (Hatakka K, Savilahti F, Pönkä A, Meurman J H, Poussa T, Näse L, Saxelin M, Korpela R. The effect of long-term consumption of a probiotic milk on the infections of children attending day care centres: a double-blind randomised trial. Submitted to BMJ in May, 2000).

### BRIEF DESCRIPTION OF THE INVENTION

It is an object of the present invention to provide means enabling the growth and activity of yeast in humans and in animals to be inhibited or reduced. This is achieved by the product, use and method of the invention, which are characterized by what is stated in the independent claims. Preferred embodiments of the invention are disclosed in the dependent claims.

The present invention is based on using specific probiotics for inhibiting yeast growth and activity, for preventing and treating diseases caused by yeast, and for relieving yeast-related symptoms in humans and in animals.

The invention thus relates to the use of the microbes Lactobacillus rhamnosus LGG, ATCC 53103, Lactobacillus rhamnosus LC705, DSM 7061, and Propionibacterum freudenreichii ssp. shermanii PJS, DSM 7067 for inhibiting yeast in humans and in animals.

For the inventive purpose the bacteria may be consumed separately or in a combination. They can be consumed as such, for example in the form of a lyophilized product, or used as an additive or an ingredient of edible products, such as dairy products and drinks. For the preparation of a combination, mixed cultures or pure cultures of each bacterium can be used. The combination can also be prepared in a unit dosage form, such as a capsule. The capsule may contain all the above bacteria, preferably in the form of lyophilized cultures, or a series of three capsules may be prepared, one for each bacterium.

The invention thus also relates to the use of the bacteria Lactobacillus rhamnosus LGG, ATCC 53103, Lactobacillus casei ssp. rhamnosus LC705, DSM 7061, and Propionibacterium freudenreichii ssp. shermanii PJS, DSM 7067 for preparing a product for inhibiting yeast.

The product can be a food industry product or pharmaceutical industry product, for example, or a health-promoting or a natural product. Preferred products include health-promoting dairy products, such as a cheese, into which the microbes are added in connection with the manufacture of the product. The microbes may also act as starters and as elements forming the structure of the cheese or other product. A second preferred product group includes pharmaceutical preparations, particularly tablets and capsules, that contain auxiliary agents and additives commonly used in these products, and possibly also other active ingredients, in addition to the above micro-organisms. Particularly preferred products include preparations for oral consumption, such as tablets and capsules, containing xylitol, in addition to LGG, LC705, and PJS. Apart from the microbes used according to the invention, the products may contain other microbes as well. The bacteria, combinations and other products disclosed herein have an effect on the growth and activity of yeast appearing in the human organ system and they prevent yeast infections. They are thus useful for preventing disorders and diseases caused by yeast, for relieving symptoms related thereto, and for general health improvement.

The invention further relates to a method for inhibiting yeast growth and for preventing or treating diseases caused by yeast or for relieving yeast-related symptoms in humans and in animals, the method comprising administering the microbes Lactobacillus rhamnosus LGG, ATCC 53103, Lactobacillus casel ssp. rhamnosus LC705, DSM 7061, and Propionibacterium freudenreichii ssp. shermanii PJS, DSM 7067 to an individual in need thereof in an amount sufficient to produce the desired result.

### DETAILED DESCRIPTION OF THE INVENTION

The present invention thus relates to the use of specific probiotics for inhibiting yeast growth and activity in the organ system of humans or animals.

Probiotics are live microbes that, when administered to humans or animals, promote the health of the host by improving the microbial balance in the intestines; in this way, or in addition to this, probiotics may have many other useful properties as well.

The most important probiotics are lactic acid bacteria, propionic acid bacteria and bifido bacteria. These belong inherently to the organ system of humans and animals. Lactobacilli are an important part of the normal bacterial flora of human organ system (Redondo-Lopez V, Cook R L, Sobel J D. Emerging role of lactobacilli in the control and maintenance of the vaginal bacterial microflora. Rev Infect Dis 1990;12:856-872). Propionic bacteria, in turn, appear on the skin and in the gastrointestinal tract (MacFarlane G T, Allison C, Gibson S A W, Cummings J H. Contribution of the microflora to proteolysis in the human large intestine. J Appl Bacteriol 1988;64:37-46). Due to their safety and health-promoting effects, probiotics are often used in foodstuffs as well.

The strains Lactobacillus rhamnosus LGG, ATCC 53103, Lactobacillus casei ssp. rhamnosus LC705, DSM 7061, and Propionibacterum freudenreichii ssp. shermanii PJS, DSM 7067 to be used in the invention have been described in the prior art.

Lactobacillus rhamnosus GG (LGG) has been described for example in U.S. Pat. No. 5,032,399, Gorbach & Goldin. The strain has been isolated from human feces, it is able to grow well in pH 3 and survives even lower pH values as well as high bile acid contents. The strain exhibits excellent adhesion to both mucus and epithelial cells. Lactic acid yield from glucose is good: when grown in MRS broth, the strain produces 1.5-2% of lactic acid. The strain does not ferment lactose. The strain employs the following carbohydrates: D-arabinose, ribose, galactose, D-glucose, D-fructose, D-mannose, rhamnose, dulcitol, inositol, mannitol, sorbitol, N-acetylglucosamine, amygdalin, arbutin, esculin, salicin, cellobiose, maltose, saccharose (slowly), trehalose, melezitose, gentibiose, D-tagatose, L-fucose, and gluconate. The strain grows well at +15-45° C., the optimum temperature being 30-37° C. Lactobacillus rhamnosus GG is deposited with the depository authority American Type Culture Collection under accession number ATCC 53103.

Lactobacillus rhamnosus GG is a natural bacterial strain in humans and its probiotic effects have been widely studied (Saxelin M. Lactobacillus GG—a human probiotic strain with thorough clinical documentation. Food Rev Int 1997;13:293-313). It remains viable in the gastrointestinal tract and is capable of temporarily colonizing the intestines (Goldin B R, Gorbach S L, Saxelin M, Barakat S, Gualtieri L, Salminen S. Survival of Lactobacillus species (strain GG) in human gastrointestinal tract. Dig Dis Sci 1992;37:121-128). LGG seems to be capable of colonizing, at least temporarily, the oral cavity as well, because the bacterium was found in the test persons' saliva for as long as two weeks after a seven-day period of LGG yoghurt consumption had ended (Meurman J H, Antila H, Salminen S. Recovery of Lactobacillus strain GG (ATCC 53103) from saliva of healthy volunteers after consumption of yoghurt prepared with the bacterium. Microbiol Ecol Health Dis 1994;7:295-298). LGG is currently added to a number of commercially available sour milk and juice products (Gefilus®).

Lactobacillus casei ssp. rhamnosus LC705 is described in greater detail in Fl Patent 92498, Valio Oy. LC705 is a gram-positive short rod occurring in chains; it is homofermentative; weakly proteolytic; grows well at +15-45° C.; does not produce ammonia from arginine; is catalase-negative; when grown in MRS broth (LAB M), the strain produces lactic acid (1.6%) having the optical activity of the L(+) configuration; the strain decomposes citrate (0.169%), thereby producing diacetyl and acetoin; the strain ferments at least the following carbohydrates (sugars, sugar alcohols): ribose, galactose, D-glucose, D-fructose, D-mannose, L-sorbose, rhamnose, mannitol, sorbitol, methyl-D-glucoside, N-acetylglucosamine, amygdalin, arbutin, esculin, salicin, cellobiose, maltose, lactose, sucrose, trehalose, melezitose, gentiobiose, D-turanose and D-tagatose. LC705 adheres weakly to mucus cells, but moderately to epithelial cells. The viability of the strain is good in low pH values and high bile acid contents. The strain survives well a salinity of 5% and fairly well a salinity of 10%. Lactobacillus casei ssp. rhamnosus LC705 is deposited with the Deutsche Sammiung von Mikroorganismen und Zellkulturen GmbH (DSM) under accession number DSM 7061.

Lactobacillus rhamnosus LC705 is used for example in the manufacture of Emmental cheese to prevent butyric acid fermentation caused by clostridia. The strain is also used in foodstuffs where it functions as an inhibitor of yeast and mold growth. In biological preservation LC705 strain is combined with Propionibacterium freudenreichii ssp. shermanii PJS (Fl 92498).

Propionibacterium freudenreichii ssp. shermanii JS (PJS) is also described in greater detail in Fl Patent 92498, Valio Oy. PJS is a gram-positive short rod; it ferments glucose, fructose, galactose and lactose; it ferments well lactate; and its optimum growth temperature is 32° C. The viability of the strain in low pH values and high bile acid contents is excellent. Propionibacterium freudenreichii ssp. shermanii JS is deposited with the Deutsche Sammiung von Mikroorganismen und Zelikulturen GmbH (DSM) under accession number DSM 7067.

In addition to the microbes to be used in accordance with the invention, the products to be manufactured may also contain other microorganisms, such as micro-organisms and probiotics contained in starters used in the dairy industry. There are numeral well-documented strains of starters that are commercially available from producers such as Hansen A/S, Denmark, and Danisco/Niesby GmbH, Germany.

The micro-organisms to be used according to the invention are cultivated using conventional methods, either as pure cultures or as different mixed cultures. The cultures can be used as such, or they can be processed as desired, for example purified, concentrated, lyophilized or finished to produce different products. The preparation of the micro-organisms to be used according to the invention is described in detail for example in publications Fl 92498 and Fl 20010157.

According to the invention, a sufficient amount of probiotics is used to produce the desired yeast inhibiting effect. The amount of each individual probiotic may therefore vary within a large range depending on for example the total amount of probiotics cells, the total daily dose and on other properties and ingredients of the product. The probiotics content in a daily dose of a combination is usually about 10⁶-10¹⁰ cfu.

According to the invention, the probiotics are suitable for consumption as such or formulated as capsules, pills, or tablets, for example, in processes conventionally applied for preparing pharmaceutical products. The probiotics to be used according to the invention can also be added to different edible products, such as foodstuffs, products of the beverage and confectionary industries, to health-promoting products, natural products, etc. Within the scope of the present invention, dairy products containing the specific probiotics, particularly cheeses and spreads, yoghurts and other sour milk products, and children's foods, juices and soups, as well as capsules, pills, and tablets are considered as preferred embodiments.

The end products are produced using conventional methods, the probiotics being added either during the process of preparing the product or afterwards, during the finishing.

The tests, which are described in greater detail in the publication, were carried out to study the effects of the probiotics to be used according to the invention on inhibiting oral yeast growth. The most common one of these yeasts, Candida albicans, which is a good representative of yeast species and widely present in the human organism, is most useful also as a model organism for other yeast species appearing in the mouth and in other parts of the organ system. The results presented in the examples show that the probiotics used according to the invention have a statistically significant reducing effect on the amount of oral yeasts. According to preliminary results it is evident that the invention is applicable in inhibiting, reducing and slowing down to a wide extent the growth and activity of yeast appearing also in other parts of the organ system, such as the intestinal tract and the urogenital area.

The invention will be described in detail with reference to the following examples, which are provided only to illustrate the invention and are not to be considered to restrict its scope of protection in any way.

### EXAMPLE 1

The Effect of Probiotics on the Occurrence of Candida albicans

The main purpose of the study was to find out whether a probiotic combination containing lacto bacilli and propionic acid bacteria could be used for reducing the occurrence of Candida albicans in the mouth. Emmental-type cheese containing live Lactobacillus rhamnosus LGG, ATCC 53103, Lactobacillus rhamnosus LC705, DSM 7061, and Propionibacterium freudenreichii ssp. shermanii PJS, DSM 7067 microbes was selected as the test material. Cheese was chosen because it is a product that belongs to an ordinary diet, it is pleasant to eat and easy to portion out in accordance with the study purpose. The cheese used as a reference was Edam cheese that did not contain the three bacterial strains in question but normal starter microbes, which were lactococci.

240 people were recruited for the study, their ages varying from 70 to 100 years.

The study was carried out as a placebo-controlled double blind test, with parallel study groups. The total study period was 19 weeks, with a run-in period of 3 weeks and 16 weeks of intervention. Use of foodstuffs containing probiotic bacteria was forbidden during the entire study. The list of forbidden foodstuffs contained e.g. Emmental and Polar cheeses, curd cheese, sour milk products containing live lactic acid bacteria, probiotics juices and different capsules and similar compressed products.

During the entire study period, the test persons adhered to their usual habits regarding oral hygiene and their normal ways of living.

Intervention: The cheese intervention continued for 16 weeks (wk). During this time half of the test persons consumed 50 g (=6-7 slices) of cheese containing probiotic bacteria daily, the other half consuming the same amount of control cheese. The cheeses were eaten unheated after the morning and evening meals, medication and teeth wash. The probiotics cheese contained 10⁷ cfu/g of L. rhamnosus LGG, 10⁷ cfu/g of L. rhamnosus LC705, and 10⁷ cfu/g of P. freudenreichii ssp. shermanii PJS. Thus the total amount of bacteria was 10⁹-10¹⁰ cfu/day.

Clinical mouth controls: Mouth controls were carried out and saliva samples taken at the old people's homes/nursing homes where the test persons lived in. The clinical checks included recording filled and removed teeth and those affected by caries (DMF). Also the condition of the periodontium was checked and classified according to the CPI index. Changes, if any, in the mucosal membrane of the mouth were recorded (including infections, discolorations, wounds, lichenoid lesions and ulcers, hyperplasia, leucoplakia, erythroplakia, and other). The clinical controls were carried out at the beginning of the study (0 wk) and at the end of it (16 wk).

Saliva samples: Saliva samples to determine yeasts, the rate of saliva secretion and buffer capacity were taken between 8 a.m. and 11 a.m. every morning. An attempt was made to take the sample always at the same time from each person. The test persons were not allowed to eat or wash their teeth for one hour before the taking of the sample. Saliva samples for yeast analysis were taken at the beginning of the study (0 wk), in the middle of it (8 wk) and at the end of it (16 wk). The rate of saliva secretion and the buffer capacity were determined from the first sample (0 wk) and the last samples (16 wk).

Yeasts were determined from saliva by taking samples from the mucosal membrane of the mouth with a cotton stick. The yeasts (mainly Candida albicans) were detected using the Dentocult CA cultivation method, in which the tubes are incubated in an incubator at 37° C. for 2 days, yeast growth being then determined semi-quantitatively on a scale of 0-3 (0=no colonies, 1=1-20 cfu/slip, 2=21-50 cfu/slip, 3>50 cfu).

Saliva secretion rate was determined by measuring both resting saliva and stimulated saliva. The limit value for hyposalivation is considered to be 0.1 ml/min of resting saliva; therefore resting saliva was collected for 15 min (1.5 ml/15 min). Stimulated saliva was collected for 5 minutes (the hyposalivation limit being 3.5 ml/5 min).

Saliva buffer capacity is associated with the rate of secretion. For this reason buffer capacity was measured using the Dentobuff test. The test was carried out on stimulated saliva, because the buffer capacity of resting saliva is always poor.

At the end of the intervention period the amount of yeast formed the primary response variable in the study. The differences in the occurrence and the amounts of yeast between the groups were tested using the Chi Square test. In addition, the occurrence of yeast or the amount thereof in the baseline situation and demographic factors (such as age, gender, prostheses), if any, were taken into account using a logistic regression analysis. In this connection, the saliva secretion rate and buffer capacity were also taken into account as explanatory factors. High saliva contents were analysed correspondingly. Changes in saliva secretion rate and the buffer capacity were described.

The results are shown in the following tables, in which A represents the group that ate the probiotics cheese, B being the control group. The number of colonies has been presented as follows:

0=no colonies

1=1-20 cfu/slip

2=20-50 cfu/slip

3=more than 50 cfu/slip

Table 1 shows the precise yeast measurement results at the beginning of the study and after 8 and 16 weeks of intervention for persons from whom all the three measurements were obtained (in group A n=92, in group B n=100). Table 2 shows classified yeast amounts at the beginning of the study and after 8 and 16 weeks of intervention for persons from whom all the tree measurements were obtained (in group A n=92, in group B n=100).

TABLE 1
Precise yeast results at the beginning of the study and
after 8 and 16 weeks of intervention

| | | A | | B | |
|---|---|---|---|---|---|
| | | n | % | n | % |
| Baseline | 0 | 27 | 29.3 | 30 | 30.0 |
| | 1 | 37 | 40.2 | 42 | 42.0 |
| | 2 | 17 | 18.5 | 14 | 14.0 |
| | 3 | 11 | 12.0 | 14 | 14.0 |
| 8 wk | 0 | 32 | 34.8 | 37 | 37.0 |
| | 1 | 37 | 40.2 | 32 | 32.0 |
| | 2 | 10 | 10.9 | 15 | 15.0 |
| | 3 | 13 | 14.1 | 16 | 16.0 |
| 16 wk | 0 | 35 | 38.0 | 33 | 33.0 |
| | 1 | 38 | 41.3 | 33 | 33.0 |
| | 2 | 11 | 12.0 | 17 | 17.0 |
| | 3 | 8 | 8.7 | 17 | 17.0 |

TABLE 2
Classified yeast contents at the beginning of the study
and after 8 and 16 weeks of intervention

| A | | B | |
|---|---|---|---|
| n | % | n | % |

0 vs. 1-4

| | | | | | |
|---|---|---|---|---|---|
| Baseline | 0 | 27 | 29.3 | 30 | 30.0 |
| | 1-4 | 65 | 70.7 | 70 | 70.0 |
| 8 wk | 0 | 32 | 34.8 | 37 | 37.0 |
| | 1-4 | 60 | 65.2 | 63 | 63.0 |
| 16 wk | 0 | 35 | 38.0 | 33 | 33.0 |
| | 1-4 | 57 | 62.0 | 67 | 67.0 |

0-1 vs. 2-4

| | | | | | |
|---|---|---|---|---|---|
| Baseline | 0-1 | 64 | 69.6 | 72 | 72.0 |
| | 2-4 | 28 | 30.4 | 28 | 28.0 |
| 8 wk | 0-1 | 69 | 75.0 | 69 | 69.0 |
| | 2-4 | 23 | 25.0 | 31 | 31.0 |
| 16 wk | 0-1 | 73 | 79.3 | 66 | 66.0 |
| | 2-4 | 19 | 20.7 | 34 | 34.0 |

0-2 vs. 3-4

| | | | | | |
|---|---|---|---|---|---|
| Baseline | 0-2 | 81 | 88.0 | 86 | 86.0 |
| | 3-4 | 11 | 12.0 | 14 | 14.0 |
| 8 wk | 0-2 | 79 | 85.9 | 84 | 84.0 |
| | 3-4 | 13 | 14.1 | 16 | 16.0 |
| 16 wk | 0-2 | 84 | 91.3 | 83 | 83.0 |
| | 3-4 | 8 | 8.7 | 17 | 17.0 |

Groups A and B were compared to each other for yeast occurrence after 8 and 16 weeks of intervention both directly and taking into account interfering factors, if any. These factors consist of age, gender, type of living, number of diagnoses, amount of medication, BMI, saliva flow rate, buffer capacity, and prosthesis, if any. The interfering factors were taken into account by using stepwise logistic regression. The group and the baseline yeast situation are forced into a model (block 1) and the interfering factors interfering on the list are taken into account stepwise (block 2; significance criterion p being less than 0.15). The results are shown in the following tables, in which table 3 shows a direct comparison in the group of relatively high yeast occurrence, i.e. classes 2-4, table 4 takes into account the interfering factors, table 5 shows a direct comparison in the group of high yeast occurrence, i.e. classes 3-4, and table 6 takes into account the interfering factors. In these tables abbreviation OR=odds ratio and abbreviation Cl for OR=confidence interval for odds ratio.

TABLE 3
Relatively high yeast occurrence (classes 2-4)

| | | | |
|---|---|---|---|
| B | p-value | OR | 95% CI for OR |

a) after 8 weeks

| | | | | |
|---|---|---|---|---|
| Group A | −0.469 | 0.178 | 0.625 | 0.316 to 1.239 |
| Initial yeast 2-4 | 2.382 | <0.0001 | 10.825 | 5.450 to 21.500 |
| Constant | −1.520 | <0.0001 | | |

b) after 16 weeks

| | | | | |
|---|---|---|---|---|
| Group A | −0.946 | 0.014 | 0.388 | 0.182 to 0.826 |
| Initial yeast 2-4 | 2.244 | <0.0001 | 9.4355 | 4.461 to 19.957 |
| Constant | −1.390 | <0.0001 | | |

TABLE 4
Relatively high yeast occurrence (classes 2-4), with interfering
factors taken into account

| | | B | p-value | OR | 95% CI for OR |
|---|---|---|---|---|---|
| Step 1 | Group A | −1.229 | 0.0082 | 0.293 | 0.118 to 0.728 |
| | Initial yeast 2-4 | 1.911 | <0.0001 | 6.757 | 2.875 to 15.884 |
| | Buffer capacity | | 0.0002 | | |
| | Buffer capacity | −2.318 | <0.0001 | 0.098 | 0.032 to 0.299 |
| | (H) | | | | |
| | Buffer capacity | −1.614 | 0.0113 | 0.199 | 0.057 to 0.694 |
| | (M) | | | | |
| | Constant | 0.481 | 0.3896 | 1.617 | |
| Step 2 | Group A | −1.378 | 0.0043 | 0.252 | 0.098 to 0.649 |
| | Initial yeast 2-4 | 1.845 | <0.0001 | 6.325 | 2.636 to 15.178 |
| | Buffer capacity | | 0.0003 | | |
| | Buffer capacity | −2.388 | <0.0001 | 0.092 | 0.029 to 0.290 |
| | (H) | | | | |
| | Buffer capacity | −1.588 | 0.0152 | 0.204 | 0.057 to 0.737 |
| | (M) | | | | |
| | Prosthesis | 1.117 | 0.0390 | 3.057 | 1.058 to 8.831 |
| | (status 1) | | | | |
| | Constant | −0.246 | 0.7165 | 0.782 | |

TABLE 5
High yeast occurrence (classes 3-4)

| | | | |
|---|---|---|---|
| B | p-value | OR | 95% CI for OR |

a) after 8 weeks

| | | | | |
|---|---|---|---|---|
| Group A | 0.029 | 0.956 | 1.029 | 0.373 to 2.837 |
| Initial yeast 3-4 | 4.022 | <0.0001 | 55.8 | 18.7 to 166.8 |
| Constant | −2.782 | <0.0001 | | |

b) after 16 weeks

| | | | | |
|---|---|---|---|---|
| Group A | −0.824 | 0.098 | 0.439 | 0.165 to 1.164 |
| Initial yeast 3-4 | 2.420 | <0.0001 | 11.244 | 4.186 to 30.208 |
| Constant | −2.142 | <0.0001 | | |

TABLE 6
High yeast occurrence (classes 3-4), with interfering factors
taken into account

| | | B | p-value | OR | 95% CI for OR |
|---|---|---|---|---|---|
| Step 1 | Group A | −1.067 | 0.0657 | 0.344 | 0.110 to 1.072 |
| | Initial yeast 3-4 | 2.221 | 0.0003 | 9.216 | 2.798 to 30.359 |
| | Buffer capacity | | 0.0370 | | |
| | Buffer capacity | −1.629 | 0.0142 | 0.196 | 0.053 to 0.721 |
| | (H) | | | | |
| | Buffer capacity | −0.551 | 0.4525 | 0.576 | 0.137 to 2.428 |
| | (M) | | | | |
| | Constant | 0.982 | 0.1136 | 0.375 | |
| Step 2 | Group A | −1.213 | 0.0437 | 0.297 | 0.091 to 0.966 |
| | Initial yeast 3-4 | 2.300 | 0.0002 | 9.974 | 2.979 to 33.396 |
| | Buffer capacity | | 0.0238 | | |
| | Buffer capacity | −1.807 | 0.0080 | 0.164 | 0.043 to 0.624 |
| | (H) | | | | |
| | Buffer capacity | −0.726 | 0.3339 | 0.484 | 0.111 to 2.109 |
| | (M) | | | | |
| | Generation of | -0.956 | 0.0991 | 0.385 | 0.124 to 1.197 |
| | women | | | | |
| | Constant | −0.154 | 0.8452 | 0.857 | |
| Step 3 | Group A | −1.475 | 0.0222 | 0.229 | 0.065 to 0.810 |
| | Initial yeast 3-4 | 2.213 | 0.0004 | 9.145 | 2.704 to 30.924 |
| | Buffer capacity | | 0.0256 | | |
| | Buffer capacity | −1.846 | 0.0086 | 0.158 | 0.040 to 0.625 |
| | (H) | | | | |
| | Buffer capacity | −0.781 | 0.3151 | 0.458 | 0.100 to 2.101 |
| | (M) | | | | |
| | Generation of | −1.371 | 0.0305 | 0.254 | 0.073 to 0.879 |
| | women | | | | |
| | Prosthesis | 1.274 | 0.1003 | 3.576 | 0.782 to 16.350 |
| | (status 1) | | | | |
| | Constant | −0.713 | 0.4344 | 0.490 | |

The results show that ageing, use of prostheses and reduced salivation are clearly associated with increased amount of yeast. Lower initial yeast amounts, in turn, were discovered with test persons who reported that they had regularly used products containing lactic acid bacteria before the study.

When examining the yeast results alone, without taking into account interfering factors, it can be seen that yeast amounts decreased more in the probiotics group than in the control group. Irrespective of whether the focus is on studying yeast occurrence (1-4), relatively high yeast occurrence (2-4), or high yeast occurrence (3-4), the results show that the proportion of those belonging to the probiotics group decreases in all of these groups as the intervention proceeds. The proportion of those belonging to the control group, in turn, does not change equally clearly. On the contrary, the proportion of those belonging to the groups of higher yeast occurrence seems even to increase in the control group.

When interfering factors are taken into account, it is observed that probiotics intervention produces a statistically significant reduction in the occurrence of both relatively high (2-4) and high (3-4) yeast amounts, as compared with the control group.

The results thus show that by using cheese containing the above probiotics, it was possible to reduce yeast amounts significantly. The present invention is thus useful for inhibiting yeast in accordance with the objectives of the invention.

1. Use of the microbes Lactobacillus rhamnosus LGG, ATCC 53103, Lactobacillus rhamnosus LC705, DSM 7061, and Propionibacterium freudenreichii ssp. shermanii PJS, DSM 7067 for preparing a product for inhibiting yeast.

2. Use of microbes according to claim 1, wherein by preparing a product of the food industry or the pharmaceutical industry, a health promoting product, or natural products.

3. Use of microbes according to claim 1, wherein by preparing a food product.

4. Use of microbes according to claim 3, wherein by preparing a dairy product, preferably cheese.

5. Use of microbes according to claim 1, wherein in that also other, conventional (starter) bacteria are used in the preparation.

6. Use of microbes according to claim 1, wherein by preparing a unit dosage form containing the microbes.

7. Use of microbes according to claim 6, wherein in that the dosage form is a preparation for oral consumption.

8. Use of microbes according to claim 7, wherein in that the dosage form is a capsule or a tablet containing xylitol, in addition to the microbes.

9. A method for inhibiting the growth of yeasts and for relieving yeast-related symptoms in animals or humans, the method comprising: administering the microbes Lactobacillus rhamnosus LGG, ATOC 53103, Lactobacillus casei ssp. rhamnosus LC705, DSM 7061, and Propionibacterium freudenreichii ssp. shermanii PJS, DSM 7061 to an individual in the need thereof in an amount sufficient to produce the desired effect.
